(19) United States
(12) Patent Application Publication
(10) Pub. No.: US 2009/0087456 A1
(43) Pub. Date: Apr. 2, 2009

(54) ADJUVANTED VACCINE

(76) Inventors: James Edward Eyles, Salisbury (GB); Margaret Gillian Hartley, Salisbury (GB)

Correspondence Address: JOHN S. PRATT, ESQ;KILPATRICK STOCKTON, LLP, 1100 PEACHTREE STREET, ATLANTA, GA, 30309, US

(21) Appl. No.: 12/066,158

(22) PCT Filed: Sep. 7, 2006
(86) PCT No.: PCT/GB06/03296

§ 371 Date: Jul. 28, 2008

(30) Foreign Application Priority Data
Sep. 7, 2005 (GB) 0518203.5
Sep. 8, 2005 (GB) 0518305.8

(51) Int. Cl. A61K 39/02

(52) U.S. Cl. 424/234.1

(57) ABSTRACT

This invention relates to new immunogenic compositions and vaccines suitable for preventing or treating tularemia.

The present invention relates to new immunogenic compositions and vaccines suitable for preventing or treating tularemia.

Francisella tularensis is one of the most infectious bacteria known to man, with inoculation or inhalation of as few as 10 organisms sufficient to cause severe disease in humans. Due to its high infectivity, together with an ease of dissemination and ability to cause severe disease and death, F. tularensis is designated as a category A agent, that is, one that is seen as a potential bioweapon.

F. tularensis is the causative agent of tularemia (also known as “rabbit fever”). Human cases of tularemia usually result from a bite from a vector such as biting flies, ticks and mosquitoes that have recently fed on an infected animal. However, there have been reported cases of infections caused by contact with dead animals, infectious aerosols, and ingestion of contaminated food and water. Hunters, veterinarians, walkers and farmers are at the greatest risk of contracting tularemia because they are likely to come into contact with infected animals. The incidence of tularemia in humans is usually low, but an increase in the number of cases is observed when there is an epidemic in the local animal reservoir.

Francisella tularensis is a member of the family Francisellaceae. There are three species within the genus Francisella, viz. F. tularensis, Francisella novicida and Francisella philomiragia. 16S ribosomal DNA sequence analysis has placed the genus Francisella as a member of the γ subclass of the proteobacteria. The F. tularensis species was originally divided into two biotypes, A and B, but recently four recognizable biotypes have been proposed. F. tularensis subspecies tularensis, previously known as Type A or subspecies nearatica, is recognised as the most virulent. It is responsible for human tularemia cases in North America and Europe and causes severe disease in mammals, especially rabbits. F. tularensis subspecies palaearctica, also known as holartica or Type B, is found in Europe, Asia and North America and is less virulent in humans than F. tularensis subspecies tularensis. F. tularensis subspecies media asiatica has been isolated from central Asia and subspecies palaearctica japonica is found only in Japan. The fourth F. tularensis subspecies is philomiragia and was originally known as the “Philomiragia” bacterium and was then renamed Yersinia philomiragia. It was finally placed in the Francisella genus on the basis of biochemical tests and cellular fatty acid analysis.

Although F. tularensis subspecies novicida and F. tularensis subspecies philomiragia are considered pathogenic to humans they pose only a small risk.

F. novicida was classified into the genus Pasteurella in 1955, but then reclassified in 1959 into the genus Francisella. It was initially considered a separate species to F. tularensis, however recently it has been proposed that it should be designated F. tularensis subspecies novicida because of the similarities between the two species. Both of these designations are utilized herein. At the genetic level, this similarity to F. tularensis is greater than 99% and the two species are chemically and antigenically very similar, demonstrating strong serological cross-reactivity. The present inventors have shown that F. novicida can be differentiated from F. tularensis on the basis of less fastidious growth requirements of F. novicida and the ability to produce acid from sucrose in F. novicida. F. novicida is fully virulent in the mouse model with a LD₅₀ of 1.76 cfu, but has reduced virulence in humans compared to F. tularensis.

In the 1940s there were attempts to make killed vaccines against tularemia consisting of whole killed cells or cell extracts, however these failed to give protection against challenge with fully virulent strains. Therefore efforts were concentrated on the production of a live vaccine. Live attenuated strains were developed in the former Soviet Union by repeatedly passaging the bacterium on media containing antiserum. Several strains were suitably attenuated for use as a vaccine and were used as such, either alone or in a mixed culture vaccine.

In 1956 a mixture of strains of Francisella tularensis were transferred from the former Soviet Union to the United States. From these strains a suitably attenuated strain was isolated and tested for safety and efficacy and was designated F. tularensis live vaccine strain (LVS). The vaccine is delivered via the scarification route using a dose of 0.06 ml and is followed by yearly boosters. Retrospective studies on the efficacy of the LVS vaccine based on laboratory acquired infections have shown that it affords good but not complete protection against typhoidal tularemia leading to a dramatic decrease in cases.

Studies using F. tularensis LVS have shown that protection is correlated with cell-mediated immunity. Protein antigens on the surface of the bacterium induce a cell-mediated response. However a large number of antigens appear to be important because there is no bias in the response towards one particular antigen. It has been found that the cytokines interleukin-1 and interferon-γ are important in providing resistance to infection. The humoral response induced by carbohydrate antigens on the bacterium also has a role in protection but can only protect against challenge by strains with reduced virulence.

Research into finding an alternative to the live vaccine has yet to yield particularly promising candidates. Subunit vaccines, so effective at treating other pathogenic diseases, has so far proved ineffective against treating tularemia. One of the primary problems of developing such a vaccine is that few immunodominant antigens of Francisella have been identified from which an effective subunit vaccine could be made. Besides, if the mode of action of infection by Francisella is that it requires a large number of epitopes, then subunit vaccines may never be able to provide an answer because by definition, subunit vaccines only have a limited number of epitopes present. Even using subcellular antigen preparations combined with a potent adjuvant such as immunostimulating complexes (ISCOMs) induced only a marginal protective response (Tarnvik et al., 1996, FEMS Immunol. Med. Microbiol. 13: 221-225).

The prior art suggests that a live vaccine is of key importance when treating intracellular bacterial infections such as F. tularensis infection (Tarnvik et al., 1996, supra). Experiments carried out using killed F. tularensis would seem to support this hypothesis: scientists found that using killed F. tularensis as a vaccine afforded no protection against virulent F. tularensis (see review article Tarnvik et al., 1989, Rev Infect Dis 11: 440-451 and also Tarnvik et al., 1996, supra, where killed LVS bacteria was used).

There is currently no effective licensed vaccine against F. tularensis and a new vaccine is required.

According to a first aspect of the present invention there is provided an immunogenic composition comprising a killed Francisella strain and one or more adjuvants.

The present inventors have found surprisingly that when a killed Francisella strain, rather than a live or live attenuated strain, is combined with one or more adjuvants, the components interact synergistically to provide a composition that is particularly suitable for immunisation purposes.

Preferably, the killed Francisella strain is a killed Francisella tularensis strain. More preferably, the strain is a strain of F. tularensis subspecies tularensis or a strain of F. tularensis subspecies palaearctica. Most preferably, the killed Francisella strain is a killed LVS (live vaccine strain). Other strains which may be used as starting material in the invention are, for example, a non-virulent strain of F. tularensis subspecies tularensis available under ATCC accession No. 6223, or various F. tularensis subspecies philomiragia strains available under ATCC accession Nos 25015-25018). Kawula et al. (2004, Appl. Environ. Microbiol. 70: 6901-6904) discloses stable insertion mutant strains of LVS using transposon-transposase complexes, which strains may also be used as starting material in the present invention. The complete genome sequence of F. tularensis subspecies tularensis (SchuS4) has been published and is available in Genbank as accession No. AJ749949.

An advantage of the present invention is that the killed Francisella strain is unable to revert back to a virulent strain, unlike live or live attenuated vaccines strains. By “killed Francisella strain” is meant a Francisella strain which is unable to replicate. The killed Francisella strain has preferably been manipulated such that its nucleic acid material will no longer be able to replicate. The bacterial proteins of a killed Francisella strain are typically inactivated such that reversion to properly folded virulent proteins is negligible.

Means of killing bacteria are well known to a person skilled in the art and include mechanical means, such as irradiation and heat activation, and chemical means. Preferably, the Francisella strain used in the invention has been killed by irradiation.

The composition may be capable of stimulating a T_{H}1 response in a host organism. For example, the adjuvant or adjuvants may direct the immune response of a host organism to the T_{H}1 response.

T-cell-mediated immunity appears to be crucial in protecting a host organism against facultative bacteria and the response by a host to virulent F. tularensis strains may be no exception.

Generally, a host organism will tailor its immune response to the type of antigen to which it is exposed. For example, if the antigen is from an extracellular parasite, the host's immune system will generally mount a T_{H}2 response. If the antigen is from an intracellular viral infection, then a T_{H}1 response is more usual. In the case of human tularaemia, the CD4 and CD8 T cells from individuals previously exposed to the disease have shown a T_{H}1 response to several homologous antigens in vitro. In a further embodiment, there is provided the use of the composition for stimulating a T_{H}1 response in a host organism.

The inventors have found that a killed Francisella strain and an adjuvant will interact synergistically in a composition to give a far more effective vaccine than either a killed Francisella strain alone or an adjuvant alone (see Examples below).

As explained above, a T_{H}1 response is considered to be important in a host's immune response to infection with Francisella. Adjuvants can be used to skew the immune response of a host organism to a particular type. In one embodiment of the invention, therefore, the adjuvant directs the immune response of a host organism to a T_{H}1 response. Preferably, the adjuvant is an ISCOM. The Examples below show that mice immunised with a killed Francisella strain and an ISCOM have a better rate of survival whether the composition is applied subcutaneously or intramuscularly.

As used herein, an “adjuvant” is a substance that enhances the immune response of a host organism to the killed Francisella strain. A substance is said to “enhance” an immune response of a host organism to an antigen (i.e. is an adjuvant) if the immune response experienced by the host organism is greater when an antigen is applied to the host organism in combination with the putative adjuvant, compared to the immune response experienced by the host organism when an antigen is applied without the putative adjuvant. Various immune cell assays can give a good indication of whether a substance is likely to be an effective adjuvant in a host organism or not (see for example, U.S. Pat. No. 6,406,705 which cites measuring the antibody forming capacity and number of lymphocyte subpopulations using a mixed leukocyte response assay and lymphocyte proliferation assay).

In a further embodiment, the immunogenic composition comprises more than one adjuvant. The adjuvant may be selected from the group consisting of alum, a CpG-motif-containing oligonucleotide and an ISCOM. For example, the composition may comprise a CpG-motif-containing oligonucleotide and alum. Alternatively, the composition may comprise a CpG-motif-containing oligonucleotide and an ISCOM. The composition may comprise at least two, three, four, five, six, seven, eight, nine, ten or more adjuvants.

The Examples below demonstrate that a composition comprising a killed Francisella strain and at least two adjuvants, when used to immunise a host organism, results in a higher rate of survival for the host organism compared to using a killed Francisella strain and only one adjuvant.

Bacterial DNA is known to have immune stimulatory effects in certain hosts that result in the activation of B cells and natural killer cells. Specifically, unmethylated CpG dinucleotides in a particular base context (CpG-motifs) have been found to stimulate the immune system in a host organism. These unmethylated CpG-motifs are common in bacterial DNA but are underrepresented in vertebrate DNA (Krieg et al., 1995, Nature 374: 546-549). Synthetic oligonucleotides containing CpG-motifs have been found to have a similar stimulatory effect when tested on human and murine leukocytes and certain CpGs have been used as a preventative against various diseases including Ebola virus, Bacillus anthracis, Listeria monocytogenes, Francisella tularensis, Plasmodium yoelli and vaccinia. The reason why the protection offered by CpG-motif oligonucleotides is so wide-ranging is because it is the innate immune response in a host organism that is triggered which is a non-specific immune response.

CpG-motif-containing DNA is thought to induce a T_{H}1 like pattern of cytokine production (Klinman et al., 1996, Proc. Natl. Acad. Sci. USA 93: 2879-83). Preferably, the composition comprises a CpG-motif-containing oligonucleotide that directs the immune response of a host organism towards a T_{H}1 response. Direction of an immune response to a T_{H}1 immune response can be assessed by measuring the levels of cytokines produced in response to the CpG-motif-containing oligonucleotide (e.g., by inducing monocytic cells and other cells to produce T_{H}1 cytokines, including IL-12, IFN-y and GM-CSF).

The present inventors have found that vaccinating a host organism with a CpG-motif-containing oligonucleotide as the sole adjuvant (in conjunction with the killed Francisella strain) does not give optimal protection to the host (see Examples below). Rather, the CpG-motif-containing oligonucleotide is preferably administered with at least one other adjuvant, for example, alum or an ISCOM, to achieve a higher rate of survival for the host.

A “CpG-motif-containing oligonucleotide” as used herein means an oligonucleotide that contains at least one unmethylated cytosine-guanine (CpG) dinucleotide sequence (that is, a 5′ cytosine followed by a 3′ guanosine) linked by a phosphate bond. The term “unmethylated CpG” refers to the absence of methylation of the cytosine on the pyrimidine ring. The term “oligonucleotide” refers to a polymeric form of nucleotides at least five bases in length. Preferably, the oligonucleotide is 6 to 100 nucleotides in length, more preferably 8 to 30 nucleotides in length. The oligonucleotide used herein may be a deoxyribonucleotide, ribonucleotide, or a modified form of either nucleotide, and includes both single and double stranded forms. Preferably, the oligonucleotide is a deoxyribonucleotide. The modification may include at least one nucleotide that has a phosphate backbone modification. For example, instead of a normal phosphodiester linkage, the phosphate backbone may have a phosphorothioate or phosphorodithioate modification (Krieg, A. M. et al., 1996, Antisense Nucl. Acid Drug. Dev. 6: 133-139; Boggs, R. T. et al., 1997, Antisense Nucl. Acid Drug. Dev. 7: 461-71). In some embodiments, the phosphate backbone modification may occur on the 5′ side of the oligonucleotide or the 3′ side of the oligonucleotide.

Nontraditional bases such as inosine and queosine, as well as acetyl-, thio- and similarly modified forms of adenine, cytidine, guanine, thymine, and uridine can also be included in the oligonucleotide, as can nonionic DNA analogs, such as alkyl- and arylphosphonates (in which the charged oxygen moiety is alkylated), as are those oligonucleotides that contain a diol, such as tetraethyleneglycol or hexaethyleneglycol, at either or both termini. The guanosine may be replaced with an analog such as 2′-deoxy-7-deazaguanosine. In one embodiment, the modification results in a nuclease resistant oligonucleotide.

The CpG-motif-containing oligonucleotide may be a linear or a circular oligonucleotide. Preferably, the CpG-motif-containing oligonucleotide is a linear oligonucleotide. “Linear” as used herein refers to an oligonucleotide which has two ends (i.e. is not circular).

Preferred oligonucleotides also do not include a CCGG quadmer or more than one CCG or CGG trimer at or near the 5′ or 3′ terminals.

The CpG-motif-containing oligonucleotide may in a preferred embodiment comprise a polynucleotide sequence having the formula:

5′-X₁X₂CGX₃X₄-3′

where C and G are unmethylated and X₁ to X₄ are any nucleotide.

CpGs have been categorised into at least three structurally distinct classes. CpG-B type CpGs (also known as ‘K-type’) encode multiple CpG motifs on a phosphorothioate backbone, and trigger the differentiation of APCs and the proliferation and activation of B cells. CpG-A type CpOs (also known as ‘D-type’) are constructued using a mixed phophodiester-phosphorothioate backbone and directly induce the secretion of IFN-α from plasmacytoid dendritic cells, which indirectly supports the subsequent maturation of APCs. CPG-C type CpGs have characteristics of both the ‘D-type’ and the ‘K-type’. They can stimulate B cells to secrete I1-6 and plamacytoid dendritic cells to produce IFN-α. They also have a phosphorothioate backbone, like the D-type but also tend to have a TCG dimer at the 5′ end.

The CpG-motif-containing oligonucleotide may be a CPG-B type oligonucleotide or a CPG-C type oligonucleotide. Preferably, the CPG-B or C type oligonucleotide is an oligodeoxyribonucleotide. The CpG-motif-containing oligonucleotide may comprise a sequence defined by one or more of the group consisting of: TCGTCGTTTTGTCGTTTTGTCGTT <SEQ ID NO: 1> (CpG7909), TCGTCGTTTTTCGGTCGTTTT <SEQ ID NO:2> (CpG10103), and TCCATGACGTTCCTGACGTT <SEQ ID NO: 3> (CpG1826).

The oligonucleotides of the present invention can be synthesized by procedures known in the art (see for example—Oligonucleotide Synthesis, Methods and Applications, erdewijn (Ed.), Rega Institute, Katholieke Universiteit Leuven, Belgium) or can be bought commercially (for example, from Sigma-Genosys [http://www.fisheroligos.com/olg_prc.htm] or Coley Pharmaceuticals). The oligonucleotides can also be prepared using known molecular cloning techniques including employing restriction enzymes (e.g. exonucleases or endonucleases).

Immunostimulating complexes (ISCOMS) are known to be highly effective adjuvants against viral agents but have been found to be less effective for delivery of antigens of intracellular bacteria such as F. tularensis.

Golovliov et al., (Golovliov et al., 1995, Vaccine 13: 261-267) found that using ISCOMS associated with the TUL4 protein of F. tularensis gave some immunizing effect, but this effect was small compared to the protective effect of a live tularaemia vaccine such as LVS.

The present inventors have found that immunizing a host organism with a composition comprising a killed Francisella strain and an ISCOM will provide some protection to the host whether the route of delivery is subcutaneous or intramuscular (see Examples below). However, a composition that comprises a killed Francisella strain, an ISCOM and a further adjuvant may result in a much higher survival rate for the host organism. In a preferred embodiment therefore, where the composition comprises an ISCOM, another adjuvant (such as a CpG-motif-containing oligonucleotide) is present.

ISCOMs and their production are well known in the art and are also commercially available (e.g. preformed ISCOMs (AbISCO-100) were used in the present invention, supplied by Isconova AB, Uppsala, Sweden (http://www.isconova.se/)). In a preferred embodiment, the ISCOMs used in the present invention are 30 to 40 nm in diameter. In a more preferred embodiment, the ISCOMs comprise saponin and contain the adjuvant Quil A.

According to a further aspect of the invention there is provided a kit comprising the composition as defined herein. The killed Francisella strain and the adjuvants may be separate components of the kit or the killed Francisella strain and the adjuvants may be present in a single composition. Where the killed Francisella strain and the adjuvants are separate components of the kit and there is more than one adjuvant, the adjuvants may be separate components or mixed together. An advantage of having the adjuvants and the killed Francisella strain separated is that different buffer conditions or storage conditions can be imposed on the separate components in order to keep them all in an optimum condition for administering to a host organism. Where the killed Francisella strain and the adjuvants are present in a single composition, reduced packaging is required and there may be associated cost benefits. In addition, if the components of the composition are in a single composition, this makes for ease of use compared to having the components separated and there is no danger of mixing the components in the wrong proportions.

Preferably, the composition is in a lyophilized form. The killed Francisella and/or the adjuvants are/is in a lyophilized form.

The kit may further include a further component comprising one or more of the following: instructions, syringe or other delivery device, or a pharmaceutically acceptable formulating solution.

The invention also provides a delivery device pre-filled with a composition of the invention.

According to another aspect of the invention, there is provided a vaccine comprising the composition as defined herein.

Preferably, the vaccine is suitable for treating tularemia where the tularemia is preferably caused by Francisella tularensis subspecies tularensis or palaeartica.

By “vaccine”, is meant a substance comprising antigenic material that can be used to stimulate the immune system of a host organism and thus confer some immune protection against one or more diseases.

In a preferred embodiment, the vaccine is suitable for treating humans. However, tularemia is not restricted to humans and, in the case of F. tularensis, the cottontail rabbit (Shylvilagus spp) is the principal mammalian target host. In another embodiment of the third aspect of the invention, therefore, the vaccine is suitable for treating a non-human animal such as a mammal. Preferably, that mammal is a cotton tail rabbit (Sylvilagus spp), a sheep, a mouse, a rat, a guinea pig, a beaver, a vole rat or a muskrat. Immunisation of mammals other than humans may not only avoid suffering for that animal but may also reduce the risk of cross-species transmission to humans.

There have been reports that injection of immunostimulatory molecules without an antigen can confer short-lived non-specific protection against some forms of tularaemia in mice (Elkins et al., 1999, J Immunology 162:2291-2298). However, the present inventors have found that such injections afford no protection against some highly virulent strains of Francisella e.g. HN63 In one embodiment, therefore, the vaccine is suitable for treating or preventing HN63 infection.

Although the vaccine of the present invention can be prepared in the many forms as described below for medicaments, e.g. creams, tablets, sprays etc., the vaccine is preferably in a lyophilized form.

Vaccines may be delivered simultaneously with other vaccines with no significant side effects or decrease in efficacy compared to when the vaccines were given separately (e.g. smallpox vaccine was commonly co-administered with Bacille Calmette-Guerin (BCG)). Advantages of co-administration include reducing production costs if the antigens for the different vaccines can be put into a single formulation, time efficiencies by the medical staff who need only administer a single formulation instead of multiple formulations, or if the co-administration is sequential, time is still saved because the medical staff do not have to wait for the patient to return again for administration of individual vaccines for individual diseases, there is also an increased likelihood that the patients will receive all the vaccines because, unlike single vaccines, there is no danger of the patient not returning, and most importantly, if the vaccines can be co-administered in a single formulation, patient suffering is decreased since only one initial administration is necessary. In one embodiment of the invention, therefore, the vaccine comprises one or more further antigens in addition to the killed Francisella strain. The further antigens may comprise part of the kit as described herein, and administration may be sequential or simultaneous. Preferably, the further antigen is not a Francisella antigen.

In a further aspect of the invention there is a provided the composition as defined herein, the kit as defined herein, or the vaccine as defined herein, for use as a medicament.

Also provided according to the present invention is use of the composition as defined herein, the kit as defined herein, or the vaccine as defined herein, as a medicament. The composition, kit or vaccine may stimulate a T_{H}1 response in a host organism. The use is preferably for the treatment of tularemia, for example caused by Francisella tularensis subspecies tularensis or palaeartica.

The composition, whether as a single composition or separated into various components (e.g. killed Francisella strain and adjuvants), may be in the form of a liquid (solution or suspension), a solid (including lyophilized compounds, a tablet, a capsule, or a dragee), a gas (including an aerosol e.g. an injectable aerosol or a spray), a gel or a cream.

The route of delivery of the medicament (for example, the composition, kit or vaccine) into a host organism may include intradermal, transdermal, subcutaneous, intravenous, intramuscular, intraarterial, intramedullary, intrathecal, intraventricular, intraperitoneal, intranasal, enteral, topical, sublingual, vaginal, rectal, oral, aural or ocular.

The composition may be suitable for topical administration, e.g. in the form of a spray, an aerosol, a gel, a cream, an ointment, a liquid, or a powder. The composition may be suitable for oral administration, e.g. in the form of a dragee, a tablet, a capsule, a spray, an aerosol, or a liquid, e.g. a syrup, a tincture (particularly when the pharmaceutical composition is solubilised in alcohol). The composition may be suitable for aural or ocular administration e.g. in the form of drops or sprays. The composition may be suitable for pulmonary administration e.g. in the form of an aerosol, a spray or an inhaler. The composition may be suitable for rectal or vaginal administration e.g. in the form of a suppository (including a pessary). The composition may be suitable for subcutaneous, intramuscular or intradermal administration e.g. in the form of an injector and/or injection. Preferably, intradermal is by a high pressure jet injector.

Gene guns or hyposprays may also be used to administer the compositions of the invention.

Preferably, the route of delivery is subcutaneous or intramuscular (see Examples). In one embodiment, where the route of delivery is subcutaneous or intramuscular and only one adjuvant is present in the composition, that adjuvant is an ISCOM. In a preferred embodiment, where the route of delivery is subcutaneous, the adjuvants are alum and a CpG-motif-containing oligonucleotide. In an alternative embodiment, where the route of delivery is intramuscular, the adjuvants are an ISCOM and a CpG-motif-containing oligonucleotide.

The composition of the invention may also be prepared in a solid form which is suitable for solubilising or suspending in a liquid. Preferably, the liquid is water or alcohol. The solid form can be a lyophilized composition or a spray freeze-dried composition. The solid form can be solubilised or suspended in liquid immediately prior to administration. Advantages of using lyophilized compositions include economical savings because of cheaper transportation costs and easier storage conditions because the compositions tend to be more stable in a lyophilized state compared to being in solution. In such cases, the composition is preferably supplied as a kit (see above) that includes all or some of the components necessary for reconstitution into a form suitable for administration to the host. The kit may contain a mixture of forms, e.g. the antigen may be in a liquid form whereas the adjuvant may be in a lyophilized state. Alternatively, all the components of the kit may be in one form e.g. all components may be in a lyophilized state.

When the composition is lyophilized, preferably, a stabilizing agent is added to the composition before lyophilization. The stabilizing agent may be peptone. For reconstitution of the composition for scarification, the composition may be reconstituted in a solution of 50% (volume per volume) glycerin in McIlvaine solution. If the lyophilized composition is intended for injection, saline is preferably used for reconstitution.

The medicament may be used prophylactically (e.g. as a vaccine) or a therapeutically (for treating a host organism that already has the disease).

The medicament may also include other components that help stabilize the composition during storage or in vivo, post-administration to the host organism. Stabilizing agents are well known in the art and include compounds such as peptone.

One or more of the adjuvants of the composition may help enhance the uptake of the antigen by antigen-presenting cells. In particular, where there are at least three adjuvants, one of the adjuvants may be mannose. Coating an antigen with mannose has been found to enhance uptake by mannose receptors on antigen presenting cells and presenting the antigen as an immune complex to take advantage of antibody and complement binding by Fc and complement receptors.

According to another aspect of the invention there is provided an antibody reactive against a killed Francisella strain as defined herein.

Methods of generating antibodies are well known in the art and include traditional methods of injecting a suitable animal with the putative antigen in order to generate polyclonal antibodies or generating monoclonal antibodies by means of hybridomas, or more modern methods such as generation of chimeric or humanized antibodies by genetic engineering means. Such means are also within the scope of the present invention.

The antibody may be a polyclonal or a monoclonal antibody, a chimeric or humanized antibody, or fragments thereof, such as Fab, F(ab′)2 and Fv, as long as it is capable of specifically binding to the required antigenic determinant (i.e. the killed Francisella strain). “Specifically binding to the required antigenic determinant” as used herein means that the antibody has to have a substantially greater affinity for the killed Francisella strain as defined herein than their affinity for other non-related antigens.

The antibodies may be employed to isolate or to identify clones expressing the epitopes responsible for the immune response generated using the killed Francisella strain in the first aspect of the invention. The antibodies may also be employed as diagnostic or therapeutic aids, amongst other applications, as will be apparent to the skilled reader.

According to a further aspect of the invention there is provided a method for producing an immunogenic composition as defined herein, comprising:
(a) inactivating a Francisella strain; and
(b) adding one or more adjuvants to the inactivated Francisella strain.

According to a final aspect of the invention there is provided a method of treating a host organism infected with or susceptible to tularemia, comprising administering to the host organism a therapeutically effective amount of the composition as defined herein, the kit as defined herein, or the vaccine as defined herein.

Embodiments of the invention will now be described by way of example only and with reference to the accompanying figures, in which:

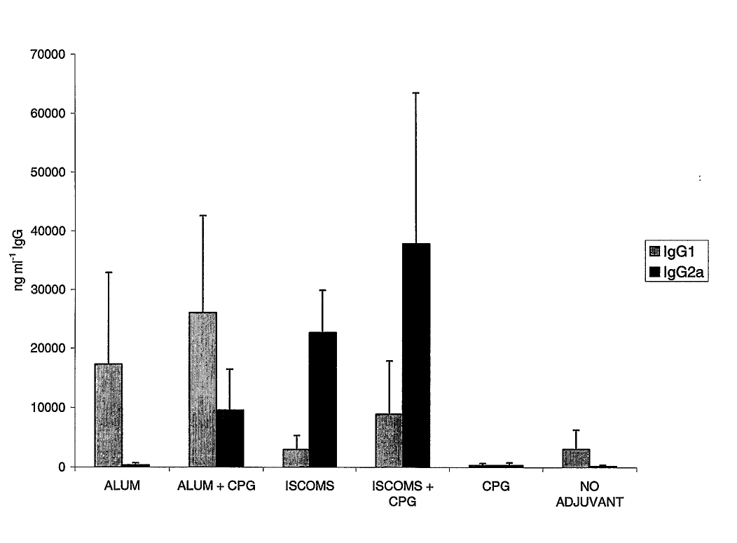

FIG. 1 is a graph showing irradiated LVS specific serum antibody titre in mice immunized by subcutaneous injection of irradiated LVS in the presence and absence of various adjuvant combinations;

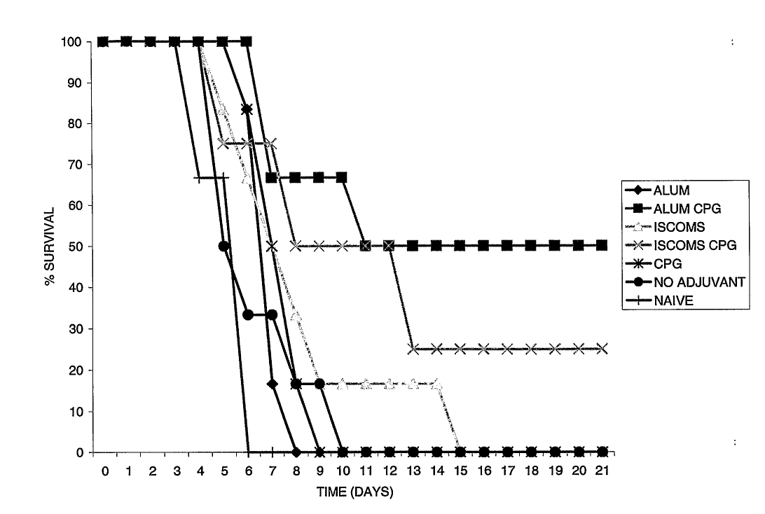

FIG. 2 is a graph showing survival against SchuS4 challenge for mice immunized by subcutaneous injection of irradiated LVS in the presence and absence of various adjuvant combinations;

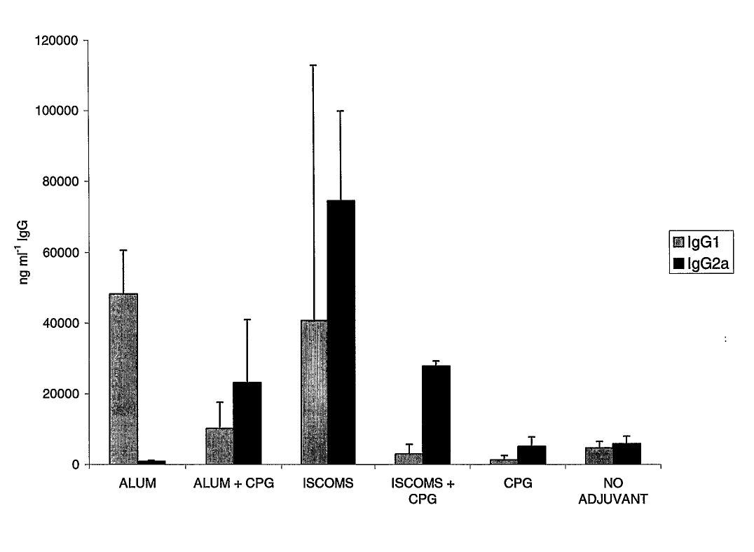

FIG. 3 is a graph showing irradiated LVS specific serum antibody titre in mice immunized by intramuscular injection of irradiated LVS in the presence and absence of various adjuvant combinations; and

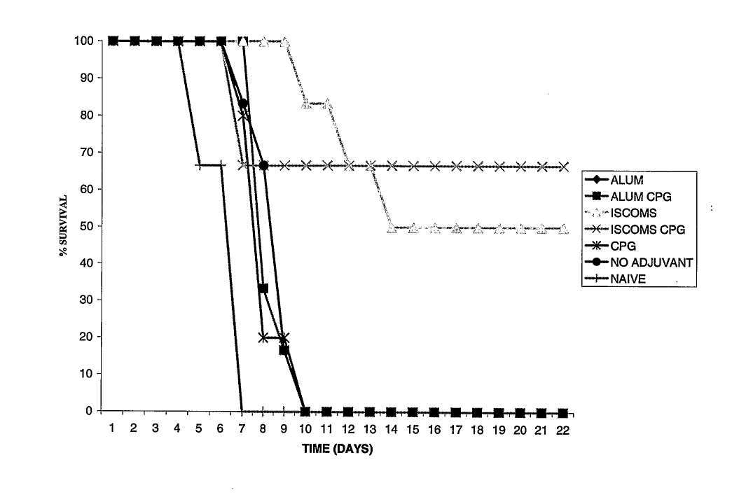

FIG. 4 is a graph showing survival against SchuS4 challenge for mice immunized by intramuscular injection of irradiated LVS in the presence and absence of various adjuvant combinations.

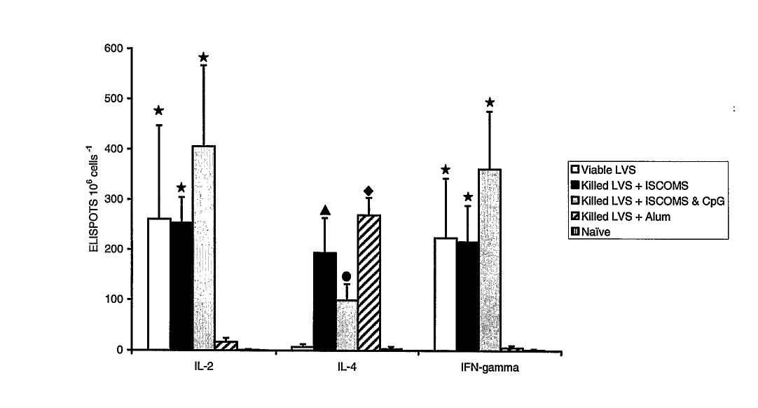

FIG. 5 is a graph showing the ELISPOT data of LVS specific cytokine secretion on day 67 following immunization of BALB/c mice on day 0, 28 and 49 with killed LVS adjuvanted with different adjuvants as described in the text. For these experiments, an additional group of mice was immunized once on day 20 with viable LVS. The mean of four individual mice per treatment group are shown, error bars represent one SD. Symbols denote statistical differences between treatment groups identified using one way analysis of variance and Student-Newman-Keuls test. ★=P<0.05 verses naïve group and mice immunized with killed LVS+Alum. ♦=P<0.05 verses naïve group and mice immunized with viable LVS and killed LVS adjuvanted with ISCOMS or ISCOMS & CpG. ▴=P<0.05 verses naïve group and mice immunized with viable LVS or killed LVS adjuvanted with ISCOMS & CpG. =P<0.05 verses naïve group and mice immunized with viable LVS.

### EXAMPLES

### Example 1 Antigens

F. Tularensis LVS was derived from an original NDBR Lot 4 vaccine ampoule produced during the 1960s. Prior to reconstitution, vaccine ampoules were stored at −20° C. according to manufacturer's instructions. Bacteria were cultured overnight at 37° C. on supplemented blood cysteine glucose agar (BCGA). LVS bacteria were resuspended in sterile PBS at a concentration of 10¹⁰ CFU ml⁻. The bacterial suspension was irradiated with 30 K greys using a C⁶⁰ source (Isotron Plc Swindon, UK). The sterility of the irradiated bacterial suspension was confirmed by overnight culture on BCGA plates. The concentration of protein in the suspension of irradiated LVS was determined using the bicinchoninic acid (BCA) assay (Pierce, Ill., USA). Irradiated bacteria were stored at −20° C. prior to use in immunization studies.

### Example 2 Adjuvants

ISCOMS (AbISCO-100) were purchased from Isomnova AB (Uppsala, Sweden). CpG 7909 was purchased from Coley Pharmaceutical Group (MA USA). Alhydrogel™ (Alum) was purchased from Brennentag (Denmark).

### Example 3 Animals

All experimentation strictly adhered to the 1986 UK Scientific Procedures Act. 6-8 week old female BALB/c (Charles River, UK) mice were used.

### Example 4 Subcutaneous Immunization

Groups of 3-6 mice were immunized by subcutaneous injection of 100 μl sterile saline containing 1.5×10⁹ CFU killed LVS (equivalent to 45 μg protein) in the presence and absence of various adjuvant combinations: (1) 260 μg Alum, (2) 260 μg Alum plus 75 μg CpG 7909, (3) 12 μg ISCOMS, (4) 12 μg ISCOMS plus 75 μg CpG 7909, (5) 75 μg CpG 7909, (6) no adjuvant. Immunized mice were boosted on day 49 with 3.5×10⁸ CFU killed LVS (equivalent to 10 μg protein) using the same adjuvant system as the primary dose.

### Example 5 Intramuscular Immunization

Groups of 3-6 mice were immunized by intramuscular injection of 100 μl sterile saline (50 μl per hind quadriceps muscle) containing 1.5×10⁹ CFU killed LVS (equivalent to 45 μg protein) in the presence and absence of various adjuvant combinations: (1) 260 μg Alum, (2) 260 μg Alum plus 75 μg CpG 7909, (3) 12 μg ISCOMS, (4) 12 μg ISCOMS plus 75 μg CpG 7909, (5) 75 μg CpG 7909, (6) no adjuvant. Immunized mice were boosted on days 33 and 49 with 3.5×10⁸ CFU killed LVS (equivalent to 10 μg protein) using the same adjuvant system as the primary dose.

### Example 6 Analysis of Serum Antibody Response

Immunized mice were bled on day 55. Serum was analyzed for the presence of anti-LVS antibodies using standard indirect ELISA methodology. Briefly, individual serum samples were aliquoted to microtitre plates pre-coated with killed LVS (5 μg ml⁻¹ in PBS). Binding of serum antibody was detected with peroxidase-labelled secondary antibody to mouse IgG1 and IgG2a (Harlan-SeraLab, Crawley Down, UK). As each subclass specific conjugate may not be equally reactive with its subclass molecule, to facilitate a comparison of one subclass titer with another, standard solutions (Harlan-SeraLab, Crawley Down, UK) of each subclass antibody in the range of 0.2-50.0 ng ml⁻¹ were assayed. The standard curves generated enabled determination of the mean concentration of each IgG subclass in serum derived from the various treatment groups.

### Example 7 Challenge Studies

Naïve and immunized mice (from examples 4 and 5) were challenged with a lethal dose of F. tularensis SchuS4 strain on day 64 of the experiment. F. tularensis SchuS4 strain was obtained from the US Army Medical Research Institute for Infectious Diseases, Maryland USA. Strain SchuS4 has a calculated MLD of <1 CFU. Mice were challenged by subcutaneous injection of 10 CFU bacteria. Subsequently, mice were monitored for a 21-day period during which time humane endpoints were strictly adhered to. The results are shown in FIGS. 1 to 4.

### Example 8 ELISPOT Assay

Selected cohorts of immunized and naïve mice, were killed on day 67 and their spleens removed for analyses of cellular responses. A group of mice immunized 47 days previously, by a single subcutaneous injection of PBS containing 10⁵ cfu live LVS, were also killed on day 67. Single cell suspensions of spleen cells were prepared in culture media (RPMI-1640) (Sigma, UK) supplemented with 10% heat inactivated foetal bovine serum (FBS) (Sigma, UK); 1% penicillin/streptomycin/glutamine (Sigma, UK) and 50 μM 2-Mercaptoethanol (2-ME) (Sigma, UK). IL-2, IFN-γ and IL-4 ELISPOT kits (BD Biosciences, Oxford UK) were used according to the manufacturer's guidelines. In brief, 96-well nitrocellulose bottomed-plates were coated with 100 μl of 5 μg ml-1 capture antibody in PBS and incubated overnight at 4° C. Free binding sites were blocked with 200 μl of supplemented RPMI for 2 hours. Spleen cell concentrations were adjusted to 5×10⁶ cells ml⁻¹ and added to the appropriated well. Analyses were always conducted on cells from individual mice in each treatment group. Cells were stimulated overnight in triplicate with either killed LVS (0.5 μg ml-1) in supplemented RPMI 1640, supplemented RPMI 1640 alone as a negative control or 2.5 μg ml-1 Concanavalin A (Sigma, Dorset, UK) as a positive control. The cells were removed from the ELISPOT plates with PBS containing 0.05% Tween-20. The site of cytokine secretion was detected with a biotin-labelled anti-mouse cytokine antibody and horseradish peroxidase-conjugated streptavidin. The enzyme reaction was developed using 3-amino-9-ethylcarbazole (AEC) substrate reagent set (Sigma, Dorset, UK). Spot forming cell numbers were determined using a dissecting light microscope (Zeiss Stemi 2000) and expressed relative to 1×10⁶ cells plated. The results are shown in FIG. 5. Statistical differences in the ELISPOT response were determined using ANOVA, Kruskal Wallis and Student-Newman-Keuls tests.

Immunization with viable LVS produced a cytokine recall response profile consistent with a biased T_{H}1 response; comparably high numbers of IFN-γ ELISPOTS and relatively low numbers of IL-4 secreting cells (FIG. 5). Conversely, injection of killed LVS with Alum produced a profile indicative of a more T_{H}2 orientated response. Immunization with killed LVS adjuvanted with preformed ISCOMS admixed with CpG gave a T-cell response that most closely matched that engendered by viable LVS; albeit with relatively greater numbers of LVS specific IL-4 secreting cells.

### Example 9 Aerosol Challenges

F. tularensis subsp. Tularensis Schu S4 or F. tularensis subsp. Holarctica HN63 was cultured in modified cysteine partial hydrolysate broth (MCPH): Difco yeast extract 6.25 g/l, casein hydrolysate 12.5 g/l, sodium chloride 6.25 g/l, dipotassium hydrogen orthophosphate 1.392 g/l, potassium dihydrogen orthophosphate 3.33 g/l, thiamine hydrochloride 2.5 mg/l cysteine hydrochloride 0.1 g/l. Final pH 6.7). After shaking for 48 hours at 37° C. the optical density of the culture was. determined to establish an approximate cell density. Serial dilutions of the culture were prepared in fresh MCPH and used to aerosol challenge immunized and naïve mice on day 67 of the experiment. Aerosol particles were generated using a Collison spray (Williamson et al., 1997, Vaccine, 15: 1079-1084). Mice were placed in a head only exposure chamber and exposed simultaneously for 10 minutes. The aerosol stream was maintained at 55% relative humidity (±4%) and 19° C. (±0.5° C.). Liquid impinger sampling was used to calculate challenge doses. Challenged mice were removed from the exposure chamber and returned to their home cages within category 3 containment. Animals were closely observed over a 21 day period for the development of symptoms. The results are shown in the following table:

TABLE 1
Protection against Tularemia in BALB/c mice immunized with viable LVS
or adjuvanted killed LVS

| | | | | Survival at day | |
|---|---|---|---|---|---|
| | | | | 21 post | Mean Time |
| Antigens | Adjuvant | Challenge | Challenge route | challenge | to death^{a} |
| — | — | 900 CFU HN63 | Aerosol | 1/5 | 7.8 ± 0.43 |
| Killed LVS | Alum | 900 CFU HN63 | Aerosol | 0/5 | 9.0 ± 1.67 |
| Killed LVS | ISCOMS | 900 CFU HN63 | Aerosol | 6/9 | 9.3 ± 0.47^{b, d} |
| Killed LVS | ISCOMS + CpG | 900 CFU HN63 | Aerosol | 10/10 | —^{b, d, f} |
| — | — | 6 CFU Schu S4 | Aerosol | 0/5 | 5 ± 0 |
| Viable LVS | — | 6 CFU Schu S4 | Aerosol | 1/8 | 8.4 ± 2.96^{b} |
| Killed LVS | ISCOMS + CpG | 6 CFU Schu S4 | Aerosol | 2/10 | 8.25 ± 1.29^{b} |

^{a}Mean (standard deviation) time to death of animals that died in days
^{b}P < 0.05 verses naïve mice receiving the same challenge (using Log Rank test)
^{d}P < 0.05 verses mice immunized with Alum adjuvanted killed LVS receiving the same challenge (using Log Rank test)
^{e}P < 0.05 verses mice immunized with CpG adjuvanted killed LVS receiving the same challenge (using Log Rank test)
^{f}P < 0.05 verses mice immunized with ISCOMS adjuvanted killed LVS receiving the same challenge (using Log Rank test)
P values <0.05 were considered significant.

Protection Against Aerosol Challenge with F. tularensis Subsp. holartica

One of the five unimmunized control mice survived an inhaled challenge of 900 CFU F. tularensis subsp. holarctica HN63 (Table 1). The remainder died with an average time to death of 7.8 days. Immunization of mice with killed LVS adjuvanted with preformed ISCOMS admixed with CpG afforded 100% (10/10 mice) protection against an inhaled challenge of 900 CFU F. tularensis subsp. holarctica HN63. Intramuscular injection of killed LVS adjuvanted with preformed ISCOMS conferred protection in 6 of 9 aerosol challenged mice and served to significantly increase time to death relative to controls. All 5 mice immunized with killed LVS adjuvanted with Alum died within 12 days of exposure to aerosolized F. tularensis subsp. holarctica HN63 (P>0.05 verses naïve controls).

Protection Against Aerosol Challenge with F. tularensis Subsp. Tularensis Schu S4

When challenged with 6 CFU aerosolized F. tularensis subsp. Tularensis Schu S4, all unimmunized control mice died with an average time to death of 5 days whereas 20% of mice immunized by intramuscular injection of killed LVS adjuvanted with preformed ISCOMS admixed with CpG with a statistical increase in time to death relative to naïve controls (Table 1). Mice immunized with viable LVS showed a similar level of resistance to aerosol challenge as mice immunized by intramuscular injection of killed LVS adjuvanted with preformed ISCOMS admixed with CpG.

1-36. (canceled)

37. An immunogenic composition comprising a killed Francisella strain and one or more adjuvants.

38. The composition of claim 37, wherein the killed Francisella strain is a killed Francisella tularensis strain.

39. The composition of claim 37, wherein the adjuvant directs the immune response of a host organism to the T_{H}1 response.

40. The composition of claim 37, wherein one or more adjuvants is selected from the group consisting of: alum, a CPG-motif-containing oligonucleotide and an immunostimulating complex.

41. The composition of claim 40, wherein the adjuvants are a CpG-motif-containing oligonucleotide and alum.

42. The composition of claim 40, wherein the adjuvants are a CpG-motif-containing oligonucleotide and an immunostimulating complex.

43. The composition of claim 40, wherein the CpG-motif-containing oligonucleotide comprises a sequence defined by one or more selected from the group consisting of: SEQ ID NO:1, SEQ ID NO:2 and SEQ ID NO:3.

44. A kit comprising the composition of claim 37.

45. A vaccine comprising the composition of claim 37.

46. A method of treating a host organism infected with or susceptible to tularemia, comprising administering to the host organism a therapeutically effective amount of the composition of claim 37.

47. The method of claim 46, wherein the composition stimulates a T_{H}1 response in a host organism.

48. The method of claim 46, wherein the composition is administered subcutaneously or intramuscularly.

49. The composition of claim 37, wherein the composition comprises an antibody reactive against a killed Francisella strain.

50. A method for producing the composition of claim 37, comprising:
(a) inactivating a Francisella strain; and
(b) adding one or more adjuvants to the inactivated Francisella strain.
